(19) United States
(12) Patent Application Publication
(10) Pub. No.: US 2013/0158633 A1
(43) Pub. Date: Jun. 20, 2013

(54) MOBILE DEVICE FOR THERAPEUTIC ACTION

(71) Applicant: SONY CORPORATION, Tokyo (JP)

(72) Inventors: Fritz HOHL, Stuttgart (DE); Ralf Boehnke, Esslingen (DE); Shin Saito, Stuttgart (DE)

(73) Assignee: SONY CORPORATION, Tokyo (JP)

(21) Appl. No.: 13/770,471

(22) Filed: Feb. 19, 2013

Related U.S. Application Data
(63) Continuation of application No. PCT/EP2011/061511, filed on Jul. 7, 2011.

(30) Foreign Application Priority Data
Aug. 19, 2010 (EP) 10 173 419.2

(51) Int. Cl. A61N 5/00

(52) U.S. Cl. CPC A61N 5/00
USPC 607/100

(57) ABSTRACT

The present disclosure relates to a mobile device for domestic use by a patient for therapeutic action on himself/herself, comprising: a millimeter wave generator that generates millimeter wave radiation, a millimeter wave antenna that emits millimeter waves generated by said millimeter wave generator in a predetermined direction, a coupling element that couples the millimeter wave antenna to the millimeter wave generator and that transmits the millimeter wave radiation generated by said millimeter wave generator to said millimeter wave antenna, a control unit that controls the millimeter wave generator, and a user interface that allows a patient to change settings of the device.

### CROSS-REFERENCE TO RELATED APPLICATIONS

The present application is a continuation of International Application No. PCT/EP2011/061511 filed Jul. 7, 2011. The present application also claims the benefit of priority under 35 U.S.C. §119 to European Patent Application No. 10 173 419.2 filed Aug. 19, 2010. The entire content of each of the foregoing applications is hereby incorporated by reference into the present application.

### FIELD OF THE DISCLOSURE

The present disclosure relates to a mobile device for domestic use by a patient for therapeutic action on himself/herself. The present disclosure deals particularly with a solution that allows users (hereinafter also called consumers or patients) themselves to mitigate pain they are experiencing.

### BACKGROUND OF THE DISCLOSURE

The most widespread approach to solve the problem of pain mitigation is to use “pain killers,” i.e. medicaments that aim at suppressing this pain (or the sensation of pain by blocking neurons/nerves . . . ). Side effects of these medicaments can include nausea and vomiting, drowsiness, itching, dry mouth, miosis, and constipation. In addition, taking these medicaments can lead to tolerance effects and to a dependence or addiction. Finally, such medicaments can be misused.

There is also another approach that is commonly called “Millimeter-Wave Therapy (MWT).” This approach uses as one component a millimeter-wave (mm-wave) generator. MWT is a therapy that is used in a clinical environment by professional practitioners on their patients. It is based on the effects that mm-wave radiation has on the human body. One of these effects is the mitigation of pain. Systems for MWT are meant to be used by professionals and are generally not designed to allow a safe and efficient operation by patients themselves.

Such methods and systems for MWT pain mitigation are, for instance, are described in “Low-Intensity Electromagnetic Millimeter Waves for Pain Therapy,” Taras I. Usichenko et al., eCAM 2006; 3(2)201-207, “Electromagnetic MillimeterWave Induced Hypoalgesia: Frequency Dependence and Involvement of Endogenous Opioids,” A. A. Radzievsky et al., Bioelectromagnetics 29:284-295 (2008), and “Suppression of Pain Sensation Caused by Millimeter Waves: A Double-Blinded, Cross-Over, Prospective Human Volunteer Study,” Alexander A. Radzievsky et al., Anesth Analg 1999;88:836-40.

U.S. Pat. No. 6,122,550 describes a device for therapeutic action on a human organism having a hermetically closed housing composed of a material which is transparent for extra high frequency radiation, a generator accommodated in the housing and generating extra high frequency radiation in the range of 36-78 GHz, and a modulator modulating the radiation which is generated by the generator with a frequency of a low frequency in the range of 0.1-9.8 Hz so as to provide a resulting radiation which is specific for a corresponding pathology of the human organism.

The company CEM TECH offers a device for millimeter wave therapy, which can be used by patients at home. It comprises a main unit including a display and buttons for program choice, detectors and wires connecting the detectors to the main unit. These wire connecting the detectors to the main unit are common USB cables so that the millimeter waves are obviously generated in the detectors as those USB cables are not able to transmit those millimeter waves. These detectors are, however, not able to generate radiation in the required frequency range (in particular at sufficiently high frequencies) with sufficient power to provide an effective treatment of a patient. Also, no particular details are disclosed how such an effective radiation shall be generated with such a device.

### SUMMARY OF THE DISCLOSURE

It is an object of the present disclosure to provide a mobile device for domestic use by a patient for therapeutic action on himself/herself, which can, for instance, be used for pain mitigation and which can be safely operated by a patient without stationary equipment and without a medical practitioner for set up and operation.

According to an aspect of the present disclosure there is provided a mobile device for domestic use by a patient for therapeutic action on himself/herself, comprising:
a millimeter wave generator that generates millimeter wave radiation,
a millimeter wave antenna that emits millimeter waves generated by said millimeter wave generator in a predetermined direction,
a coupling element that couples the millimeter wave antenna to the millimeter wave generator and that transmits the millimeter wave radiation generated by said millimeter wave generator to said millimeter wave antenna,
a control unit that controls the millimeter wave generator, and
a user interface that allows a patient to change settings of the device.

According to another aspect of the present disclosure there is provided a mobile device for domestic use by a patient for therapeutic action on himself/herself, comprising:
a generation means for generating millimeter wave radiation,
an antenna means for emitting millimeter waves generated by said generation means in a predetermined direction,
a coupling means for coupling the antenna means to the generation means and for transmits the millimeter wave radiation generated by said generation means to said antenna means,
a control means for controlling the generation means, and
an interface means for allowing a patient to change settings of the device.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF DRAWINGS

A more complete appreciation of the disclosure and many of the attendant advantages thereof will be readily obtained as the same becomes better understood by reference to the following detailed description when considered in connection with the accompanying drawings, wherein:

FIG. 1 shows a schematic block diagram of a device according to the present disclosure,

FIG. 2 shows a first embodiment of a device according to the present disclosure,

FIG. 3 shows a second embodiment of a device according to the present disclosure,

FIG. 4 shows a third embodiment of a device according to the present disclosure,

FIG. 5 shows a fourth embodiment of a device according to the present disclosure,

FIG. 6 shows a fifth embodiment of a device according to the present disclosure,

FIG. 7 shows a sixth embodiment of a device according to the present disclosure,

FIG. 8 shows a seventh embodiment of a device according to the present disclosure, and

FIG. 9 shows an eighth embodiment of a device according to the present disclosure.

### DESCRIPTION OF EMBODIMENTS

Referring now to the drawings, wherein like reference numerals designate identical or corresponding parts throughout the several views.

The present disclosure is based on the idea to provide a device for domestic use by a patient for therapeutic action on himself/herself, which can be safely operated by a patient without stationary equipment and without a medical practitioner for set up and operation. For this purpose, the elements of the device are included in a mobile device and a user interface is provided that allows a user to change settings of the device by himself/herself without the help of an expert or medical practitioner. The elements of the device are arranged and adapted such that domestic use is possible, but nevertheless an effective treatment is safely possible, i.e. the radiation can be generated with sufficient power and at a sufficiently high frequency.

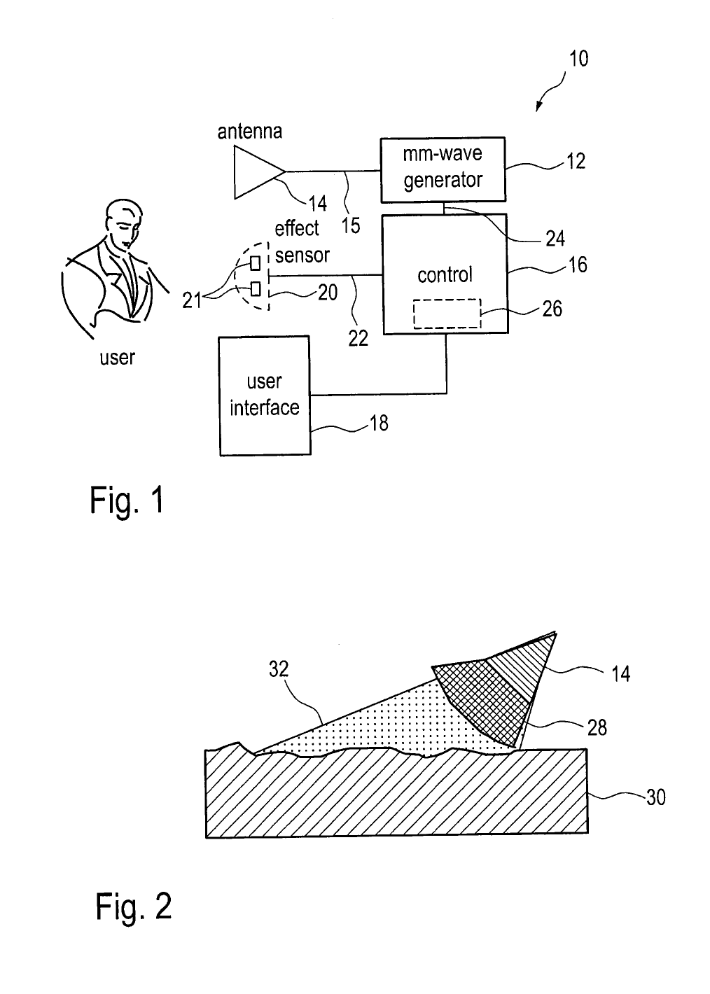

FIG. 1 schematically shows an embodiment of a device 10 according to the present disclosure that can be used by patients (i.e. end customers). The device 10 comprises a millimeter wave generator 12 that generates millimeter wave radiation, a millimeter wave antenna 14 that emits millimeter waves generated by said millimeter wave generator in a predetermined direction, a control unit 16 that controls the millimeter wave generator, and a user interface 18 that allows a patient to change settings of the device. Optionally, an effect sensor unit 20 comprising one or more sensor elements 21 that sense effects of the therapeutic action on the patient is additionally provided.

The millimeter wave antenna 14 is coupled to the millimeter wave generator 12 through a coupling element 15, which is able to transmit the millimeter wave radiation generated by said millimeter wave generator 12 to said millimeter wave antenna 14, i.e. which is able to transmit the generated radiation at a suitable (sufficiently high) frequency and with a (sufficiently high) power level. Such a coupling element 15 may, for instance, be implemented as a waveguide or a high frequency cable. Just to give an example, said coupling element is adapted in an implementation to transmit radiation in a frequency range of 30 to 50 GHz and at a power density in the range from 1 to 30 mW/cm².

The control unit 16 controls the operation of the device 10 in terms of safety and overall function. Upon the user having mounted the device at the foreseen position, the user starts the treatment, e.g. by pressing a corresponding button of the user interface 18. Upon this event the control unit 16 starts the treatment by parameterizing the mm-wave generator 12 and by switching on the radiation generation at the mm-wave generator 12.

Optionally, the control unit 16 can optimize the treatment towards the user by employing the effect sensor unit 20 that measures one or more values from the body of the user (e.g. a heart rate monitor that measures the heart rate of the user; more examples will be explained below). Upon receiving this value, the control unit 16 can either change one or more parameters of the mm-wave generator 12 until some optimum is reached or a certain duration value for the treatment is reached. In the latter case, the treatment is then stopped by switching off the mm-wave generator 12. Those optional feedback loops 22, 24 are also explained below in more detail. It should be noted that the connection 24 is not only part of the feedback loop but the general connection between control unit 16 and the mm-wave generator 12.

Even if the optional feedback loops 22, 24 are not present, in another embodiment the control unit 16 monitors the application time and ends the treatment session after some Maximum Application Session Duration value is reached by switching off the mm-wave generator 12. A new treatment session cannot be started by the user before a Minimum Treatment Pause Duration is over. An alternative in this case to switching the generator 12 off is to recommend not to start treatment again for a certain time or to remind the user to start the next treatment not before the Minimum Treatment Pause Duration is over.

In another embodiment, the user can stop the treatment session at any time. In this case the user can continue the treatment session for the rest of the Maximum Application Session Duration if the time difference between stopping and re-starting the treatment session is less than the Minimum Treatment Pause Duration. If the time difference is larger, the entire Maximum Application Session Duration time can be used.

A treatment typically consists of more than one treatment session. In one of the embodiments, the control unit 16 includes a counter 26 for counting the sessions that belong to one treatment. In such or in another embodiment, the control unit 16 is configured to inform the user about done and upcoming sessions of a treatment.

The mm-wave generator 12 generates the mm-wave radiation (in one of the embodiments, mm-wave generation is in the range between 30 and 300 GHz) and sends it to the antenna 14. The generator 12 is controlled by the control unit 16 that is able to switch the mm-wave generator 12 on and off and to set the parameters of the radiation generation (i.e. to change the settings of the generator 12). Parameters will be explained in detail below.

Power is generally supplied to the device through an external power supply, e.g. from an external power plug the mains voltage is supplied. In another embodiment, internal power supply means (e.g. a battery or accumulator) may be provided.

The antenna 14 actually radiates the mm-wave radiation generated by the mm-wave generator 12. Typically, the antenna 14 has an opening angle that disseminates the radiation over the radiation area 32 (also called illumination area) determined by the opening angle and the distance of the antenna 14 to the skin 30. This area determines the area of application of the radiation and the power density per area. In order to ensure a maximum power density for a safe operation of the device, typically a mechanical means, such as a distance holder 28, will ensure a minimum distance between the antenna 14 and the patient's skin 30 as shown in FIG. 2.

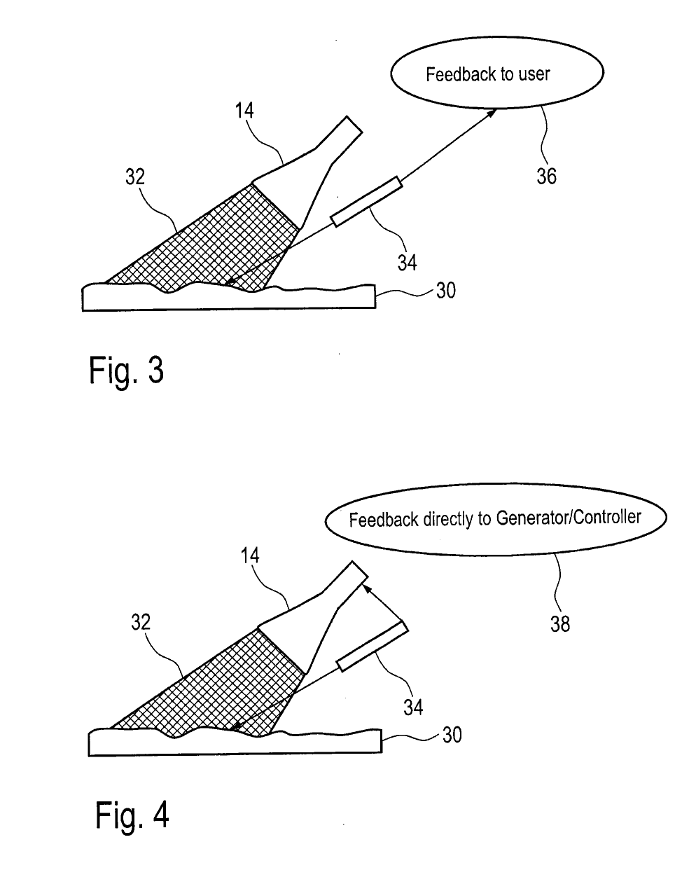

In another embodiment, as shown in FIG. 3, the mechanical distance holder is replaced (or complemented) by a distance measurement unit 34, which is able to measure the current distance between the antenna 14 and the patient's skin 30 in order to recommend, via a feedback 36 to the user, e.g. through the user interface 18, an optimum range and/or to recommend a change or the actual distance. The distance measurement unit 34 can include electronic or optical distance measurement means, e.g. based on ultrasound or laser light reflection. Such a feedback can, for instance, be an optical or acoustical signal, or a simple sign or instruction on a display.

In another embodiment, as shown in FIG. 4, the intensity of the radiation may be adaptively controlled by the distance measurement value. For this purpose the distance measurement unit 34 provides a feedback 38 to the control unit 16 and/or directly to the generator 12, which then adjust the parameters of the generator 12, e.g. the level or intensity of the generated radiation.

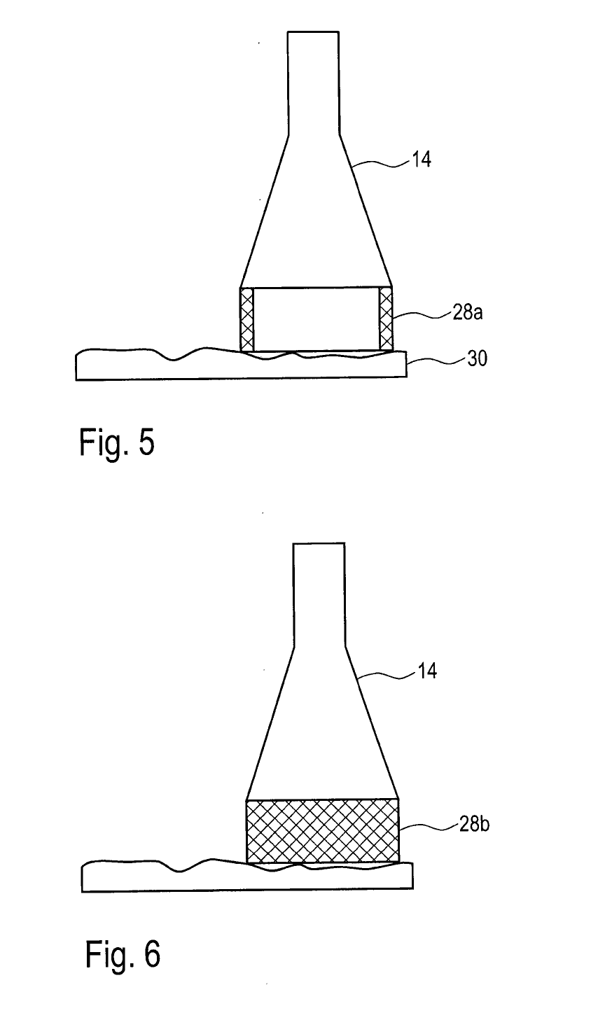

In another embodiment, as shown in FIG. 5, the distance holder 28 may comprise a ring 28a made of rigid material to ensure a controlled distance to the skin and e.g. confine the radiation within the ring.

In another embodiment, as shown in FIG. 6, the mechanical distance holder 28 comprises a rigid (integral) body 28b fully covering the antenna aperture. Preferably, though not necessarily, the body is made of a low-loss dielectric material, which ensures uniformity of the applied radio wave intensity and control of the application point (i.e. in case of a focusing dielectric lens) or which serves as a means to make the radiation transition into the skin more efficient by reducing coupling/reflection losses. Such transition layers and the specific design, i.e. thickness adapted to specific frequency and material type to achieve low loss transitions are generally known (for other applications), for instance from U.S. Pat. No. 7,371,217 B2 (incorporated by reference herein in its entirety).

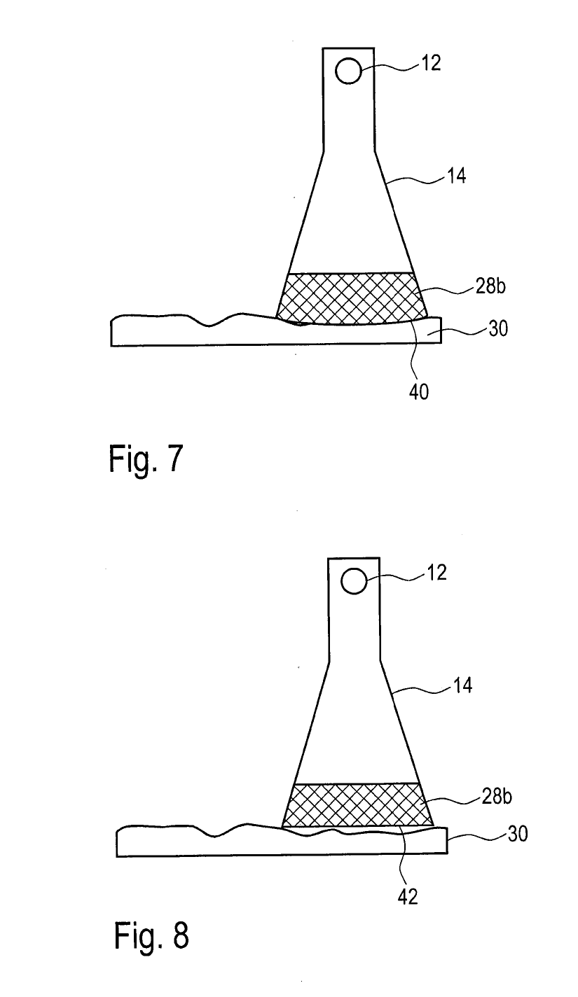

Depending on the final shape of the distance holder 28 the dielectric interface layer 28b may also be embedded directly into a waveguide (or (horn) antenna or rectangular waveguide or cylindrical waveguide) hosting the mm-wave generator 12 and then attached to the skin directly. This is depicted in FIGS. 7 and 8, where two different surfaces towards the skin 30 are depicted, in particular a curved surface 40 (FIG. 7) and a flat surface 42 (FIG. 8).

The optional effect sensor unit 20 senses the effect of the treatment on the user's body. It returns the measured data to the control unit 16 via a feedback look 22. For the concrete implementation of the effect sensor unit multiple embodiments exist, i.e. the effect sensor unit 20 may comprise one or more of the following sensor elements 21.

In an embodiment the effect sensor unit 20 may comprise a brain or heart wave pattern sensor that measures the patient's brain and/or heart wave pattern, in particular an EEG and/or EKG (assuming a unique correlation of brain or heart wave pattern with respect to pain intensity or pain relief profile is feasible).

In other embodiments the effect sensor unit 20 may comprise one or more of a temperature sensor that measures the patient's body temperature, a heart rate monitor that measures the patient's heart rate, a blood pressure sensor that measures the patient's blood pressure, a breath sensor that measures the patient's rate and/or depth of breathing, a skin moisture sensor that measures the moisture of the patient's skin and/or a skin tension sensor that measures the tension of the patient's skin. Preferably, though not necessarily, the sensor elements are arranged and/or adapted such that the measured parameter is measured at the place of irradiation of the radiation on the patient's skin.

Still further, in an embodiment an eye movement sensor that measures the eye movement pattern is provided.

The user interface 18 bundles all interaction means of the device with the user (apart from the mm-wave radiation), i.e. all displays, buttons and/or other interface elements are located in the user interface 18. Whenever another unit wants to provide some information to the user or get input from the user, it addresses the user interface 18.

There are a number of parameters of the mm-wave generator 12, which may be controlled by the control unit 16, either automatically or in response to an input from the user. These parameters can be selected independently during treatment, or as a group of values, in parameter sets which, in turn, could be activated/de-activated before or during the treatment. These parameters include:
center frequencies to be used simultaneously;
instead of single frequencies, also noise can be used emitted and applied;
bandwidths to be used around the center frequencies, single frequency, sweeping frequencies and sweeping cycles in case of swept systems;
different pulse forms, if a pulsed signal is used.

A preferred embodiment of the proposed device aims at optimizing the application of mm-wave radiation towards a user by measuring the effect of the treatment using the above described effect sensor unit 20. As a result of the method reacting on the measured value the treatment is slightly modified until a certain optimum is measured. This optimization could either address on or more of the parameters of the mm-wave generator 12 (e.g. single frequencies, or usage of noise, bandwidth, etc.) or the duration of a treatment session or the number of repetitions of the treatment.

A possible implementation of this mechanism uses a heart rate monitor as an effect sensor element 21 and the used single frequency of the mm-wave generator 12 as the actor. Here it is assumed that the patient feels the applied mm-wave radiation as soon as it has an effect on him/her. As a consequence, the heart rate of the user will slightly be raised. This effect can be used in order to sweep the frequency of the mm-wave generator 12 until the “window” of frequencies is found that the user feels. Finally, the center of this window is then subsequently used for the treatment. At the same time the heart rate monitor can be used as a safety means that switches off the mm-wave generator 12 upon measuring critical levels of heart rate.

Due to the high nerve ending density, acupuncture points are the preferred treatment locations for mm-wave radiation for pain mitigation. Most acupuncture points do, however, not lie at locations that are obvious to the untrained eye. Therefore, applying mm-wave radiation at acupuncture points by laymen requires some support. This can be achieved by various means.

In an embodiment, the device may be adapted to be mounted to the patient's body in an area where one or more acupuncture points are covered by the radiation area if fixed in a natural way. This embodiment could, for instance, be a watch-like device that covers acupuncture points close to the wrist.

In another embodiment, the antenna 14 may be designed in a way that the radiation area covers (also) the targeted acupuncture point with the required power density (i.e. by chosing a wider beam) irrespective of whether the device moves a little bit when worn (i.e. taking the backlash into account).

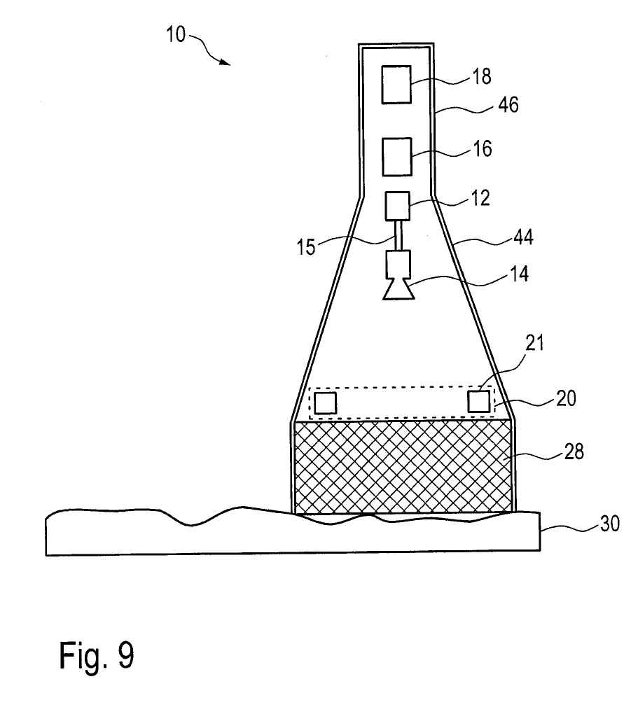

Generally, the device according to the present disclosure is configured and adapted for use by patients themselves at home, i.e. without the need of a medical practitioner to set up and operate the device and without the need of stationary equipment. The device is thus generally configured as a mobile device that can be operated and held by a patient (or a supporting person) or that can be attached to the patient's body. In another embodiment, as depicted in FIG. 9, all elements of the device are accomodated in a housing 44, and said housing includes a handle 46 for holding the device in hand. The mobile device may also be adapted to be attachable to a stand or tripod or any other type of holding means.

In summary, the device according to the present disclosure provides a patient with the ability to mitigate pain without the need for medicaments. The device is easy to handle and can be safely applied by end users at home.

The disclosure has been illustrated and described in detail in the drawings and foregoing description, but such illustration and description are to be considered illustrative or exemplary and not restrictive. The disclosure is not limited to the disclosed embodiments. Other variations to the disclosed embodiments can be understood and effected by those skilled in the art in practicing the claimed disclosure, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A modile device for domestic use by a patient for therapeutic action on the patient, comprising:
a millimeter wave generator that generates millimeter wave radiation,
a millimeter wave antenna that emits millimeter waves generated by said millimeter wave generator in a predetermined direction,
a coupling element that couples the millimeter wave antenna to the millimeter wave generator and that transmits the millimeter wave radiation generated by said millimeter wave generator to said millimeter wave antenna,
a control unit that controls the millimeter wave generator, and
a user interface that allows a patient to change settings of the device.

2. The device as claimed in claim 1, further comprising one or more sensor elements that sense effects of the therapeutic action on the patient.

3. The device as claimed in claim 2, wherein said one or more sensor elements includes:
a temperature sensor that measures a body temperature of the patient,
a heart rate monitor that measures a heart rate of the patient,
a blood pressure sensor that measures a blood pressure of the patient,
a breath sensor that measures a rate and/or a depth of breathing of the patient,
a skin moisture sensor that measures a moisture of the patient's skin, and/or
a skin tension sensor that measures a tension of the skin of the patient, including at a place of irradiation of the radiation.

4. The device as claimed in claim 2, wherein said one or more sensor elements include a brain or heart wave pattern sensor that measures a brain and/or a heart wave pattern of the patient, including using an EEG and/or EKG.

5. The device as claimed in claim 2, wherein said one or more sensor elements include an eye movement sensor that measures an eye movement of the patient.

6. The device as claimed in claim 2, further comprising a feedback loop between the one or more sensor elements and the control unit and/or the generator,
wherein said control unit is operable to change current settings of the device, including at least of the generator, based on one or more signals measured by one or more sensing elements.

7. The device as claimed in claim 1, wherein said control unit is operable to control a maximum duration of a therapeutic action and/or a minimum duration of a therapeutic pause.

8. The device as claimed in claim 7, wherein said control unit includes a counter that counts a number of subsequent sessions of a same therapeutic action,
wherein said user interface is operable to provide information regarding a number of already done and/or upcoming sessions.

9. The device as claimed in claim 1, further comprising a distance holder that ensures a minimum distance between the millimeter wave antenna and an irradiated area of the patient's skin.

10. The device as claimed in claim 9, wherein said distance holder includes a ring of rigid material that confines the radiation to a predetermined area within said ring.

11. The device as claimed in claim 9, wherein said distance holder includes an integral body made of a dielectric material.

12. The device as claimed in claim 1, further comprising a distance measurement unit that measures a distance between the millimeter wave antenna and an irradiated area of the patient's skin.

13. The device as claimed in claim 12, wherein said user interface is operable to recommend an optimal distance and/or a change of an actual distance based on the measured distance.

14. The device as claimed in claim 12, wherein said control unit is operable to change current settings of the device, including at least of the generator, based on the measured distance.

15. The device as claimed in claim 1, further comprising a housing that houses all elements of the device, said housing including a handle to hold the device for hand-held use.

16. The device as claimed in claim 1, wherein one or more settings of the millimeter wave generator that are changeable by said control unit and/or said user interface include one or more of a number and a frequency of center frequencies of the generated radiation, an emission of monochromatic or polychromatic radiation or of noise, a bandwidth around one or more of the center frequencies, sweeping frequencies and sweeping cycles, and/or pulse forms in a case of pulsed radiation.

17. The device as claimed in claim 16, wherein said millimeter wave generator is operable to generate radiation in the range from 10 to 500 GHz and/or in the range from 30 to 300 GHz.

18. A modile device for domestic use by a patient for therapeutic action on the patient, comprising:
a generation means for generating millimeter wave radiation,
an antenna means for emitting millimeter waves generated by said generation means in a predetermined direction,
a coupling means for coupling the antenna means to the generation means and for transmits the millimeter wave radiation generated by said generation means to said antenna means,
a control means for controlling the generation means, and
an interface means for allowing a patient to change settings of the device.
